(19) United States
(12) Patent Application Publication
(10) Pub. No.: US 2014/0364955 A1
(43) Pub. Date: Dec. 11, 2014

(54) ORTHOPEDIC SHOCK DAMPER SYSTEM

(71) Applicant: Bradley R. Smith, Columbus, MI (US)

(72) Inventor: Bradley R. Smith, Columbus, MI (US)

(21) Appl. No.: 14/300,285

(22) Filed: Jun. 10, 2014

Related U.S. Application Data
(60) Provisional application No. 61/956,427, filed on Jun. 10, 2013.

(51) Int. Cl. A61F 2/38

(52) U.S. Cl. CPC A61F 2/38; A61F 2/3859; A61F 2/389; A61F 2002/30563
USPC 623/20.28

(57) ABSTRACT

A shock absorbent system for a joint prosthesis includes a shock absorbent material. The shock absorbent system may be used in both hip and knee joint prostheses. In one example, a knee joint prosthesis includes a tibial plate, a femoral component having a moveable condyle in contact with a shock absorbent material, wherein movement of the moveable condyle deforms the shock absorbent material, and a tray insert disposed between the tibial plate and the moveable condyle. The moveable condyle deforms the shock absorbent material under compressive forces between the tibial plate and the femoral component, thereby absorbing impact loads

### CROSS-REFERENCE TO RELATED APPLICATIONS

This application claims the benefit of U.S. Provisional Application No. 61/956,427 filed Jun. 10, 2013. The disclosure of the above application is incorporated herein by reference.

### FIELD

The present disclosure relates to an orthopedic shock damper system, and more particularly to a shock damper system used in artificial hip and knee prosthetic joint systems.

### BACKGROUND

The statements in this section merely provide background information related to the present disclosure and may or may not constitute prior art.

In the past, surgical techniques and prostheses have been developed to replace damaged or diseased joints. For example, acetabulofemoral joint or hip joint as well as knee joint replacement surgeries have become relatively common. A hip joint replacement surgery may be a total hip arthroplasty where both the femoral head and acetabulum are replaced or a partial hip arthroplasty where only the femoral head is replaced. A hip joint prosthesis typically includes a femoral component and an acetabular cup. The femoral component is a one-piece or two-piece femoral stem with attached prosthetic femoral head (the ball). Bone is removed from the femur, or thigh bone, so as to accept the stem of the femoral component. The acetabular cup may be either a one-piece or modular component and is sized to receive the ball of the femur component. A plastic or ceramic insert may be disposed between the femoral ball and the acetabular cup. The bone and cartilage are removed from the acetabulum so as to accept the acetabular cup.

Knee joint replacement surgery may a total knee arthroplasty where the weight bearing surfaces of the knee joint are replaced with a knee joint prosthesis or a partial knee arthroplasty where compartments or segments of the weight bearing surfaces are replaced. Generally, the bone is removed from the tibia to form a tibial plateau. A tibial component having a one-piece tibial stem and a tibial platform is then attached to the tibial plateau. An insert component, typically made from polyethylene plastic, is attached to the tibial platform. A femoral component that includes artificial femoral condyles replace either one or both of the condyles of the femur.

Current hip and knee prostheses are relatively inelastic, with impact loads being directly transferred between the pelvis to the femur and between the femur and the tibia. Attempts have been made to increase the shock absorption characteristics of hip and joint prostheses. For example, U.S. Pat. No. 5,839,107 by Nassar et al. discloses a shock absorbent prosthetic hip that includes a mechanical piston and spring section. However, this prosthesis increases the complexity and cost of the hip prosthesis while simultaneously increasing the chance of mechanical failure within the shock absorbent section. U.S. Pat. No. 5,735,905 by Parr discloses an elastomeric component for a hip prosthesis disposed between the femoral stem and the ball. However, this prosthesis relies on a complicated femoral component with an injected molded elastomer. Accordingly, there is a need in the art for an improved shock absorption system for both hip and knee prostheses that absorbs impact loads while minimizing complexity of the components.

### SUMMARY

A shock absorbent system for a joint prosthesis is provided. The shock absorbent system may be used in both hip and knee joint prostheses. In one example of the present invention, a hip joint prosthesis is provided. The hip join prosthesis includes a shock absorbent material disposed between an acetabular cup and an insert. In one example of the present invention, a knee joint prosthesis is provided. The knee joint prosthesis includes a tibial plate, a femoral component having a moveable condyle in contact with a shock absorbent material, wherein movement of the moveable condyle deforms the shock absorbent material, and a tray insert disposed between the tibial plate and the moveable condyle. The moveable condyle deforms the shock absorbent material under compressive forces between the tibial plate and the femoral component, thereby absorbing impact loads. A honey comb pattern may be formed on all areas of the hip joint prosthesis and the knee joint prosthesis that meet the bone of the recipient of the prostheses so as to facilitate bone growth. The characteristics of the shock absorbent material may be gender and weight specific and the prostheses are custom made to a recipient using a bone mapping procedure.

In one aspect of the present invention, the femoral component includes a base support that defines a slot, and the moveable condyle is disposed within the slot.

In another aspect of the present invention, the base support includes a back plate, and the shock absorbent material is in contact with the moveable condyle and the back plate.

In another aspect of the present invention, the back plate includes a stepped portion having a treated surface configured to engage a matching slot on a femur.

In another aspect of the present invention, the back plate includes a stem configured to be secured within a femur.

In another aspect of the present invention, the shock absorbent material is comprised of a material having a honeycomb cross-section.

In another aspect of the present invention, a deformation characteristic of the shock absorbent material is tuned for a recipient of the knee joint prosthesis by adjusting a thickness of walls of the honeycomb material and/or a distance between the walls of the honeycomb material.

In another aspect of the present invention, a shock absorbent pad disposed between the tibial plate and the tray insert.

In another aspect of the present invention, the shock absorbent pad completely covers a surface of the tibial plate and a surface of the tray insert.

Further aspects and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a side, partial cross-section of a shock absorbent hip joint prosthesis according to the principles of the present invention;

FIG. 2 is an enlarged, perspective view of a honeycomb structure used for a shock absorbent material;

FIG. 3 is a front, partial cross-section of a shock absorbent knee joint prosthesis according to the principles of the present invention;

FIG. 4 is a cross-section of the shock absorbent knee joint prosthesis viewed in the direction of arrows 4-4 in FIG. 3; and

FIG. 5 is a cross-section of the shock absorbent knee joint prosthesis viewed in the direction of arrows 5-5 in FIG. 3.

### DETAILED DESCRIPTION

The following description is merely exemplary in nature and is not intended to limit the present disclosure, application, or uses.

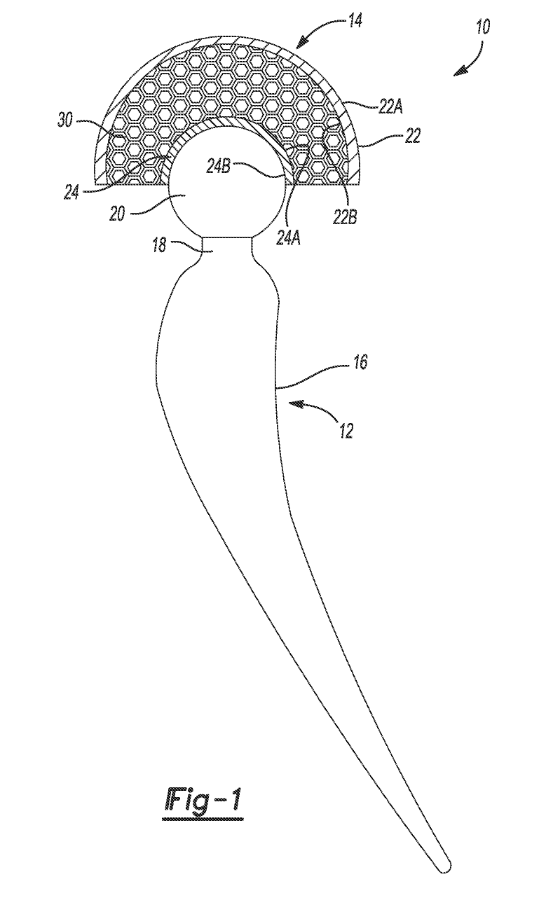

With reference to FIG. 1, a shock absorbent hip joint prosthesis according to the principles of the present invention is generally indicated by reference number 10. The hip joint prosthesis includes a femoral component 12 secured to a femur (not shown) and an acetabular component 14 secured to an acetabulum in a pelvis bone (not shown). The femoral component 12 includes a femoral stem 16 configured to be secured within the femur. The femoral stem 16 may be cemented to the femur or uncemented. The femoral stem 16 is typically metal and may have surface treatments to facilitate adhesion to the femur and/or bone growth. A neck 18 extends out from the femoral stem 16. A femoral ball 20 is secured to the neck 18. The femoral ball 20, the neck 18, and the femoral stem 16 may be formed of a single unitary piece of metal. Alternatively, the femoral ball 20 may be a separate piece secured to the neck 18. In this example, the femoral ball 20 may be either metal, plastic, or ceramic. It should be appreciated that the femoral component 12 may have various other shapes, sizes, and configurations without departing from the scope of the present invention.

The acetabular component 14 includes an acetabular cup 22 configured to be secured to the acetabulum of the pelvis. The acetabular cup or shell 22 is generally hemispherical with a porous outside surface 22A to promote bone growth and an inside surface 22B. The acetabular cup 22 is preferably made from metal.

An insert 24 is disposed within the acetabular cup 22. The insert 24 is generally hemispherical and includes an outer surface 24A and an inner surface 24B. The inner surface 24B is sized to receive the femoral ball 20 to form a ball joint. The insert 24 is preferably made from a high density polyethylene plastic or ceramic material to reduce friction between the insert 24 and the femoral ball 20.

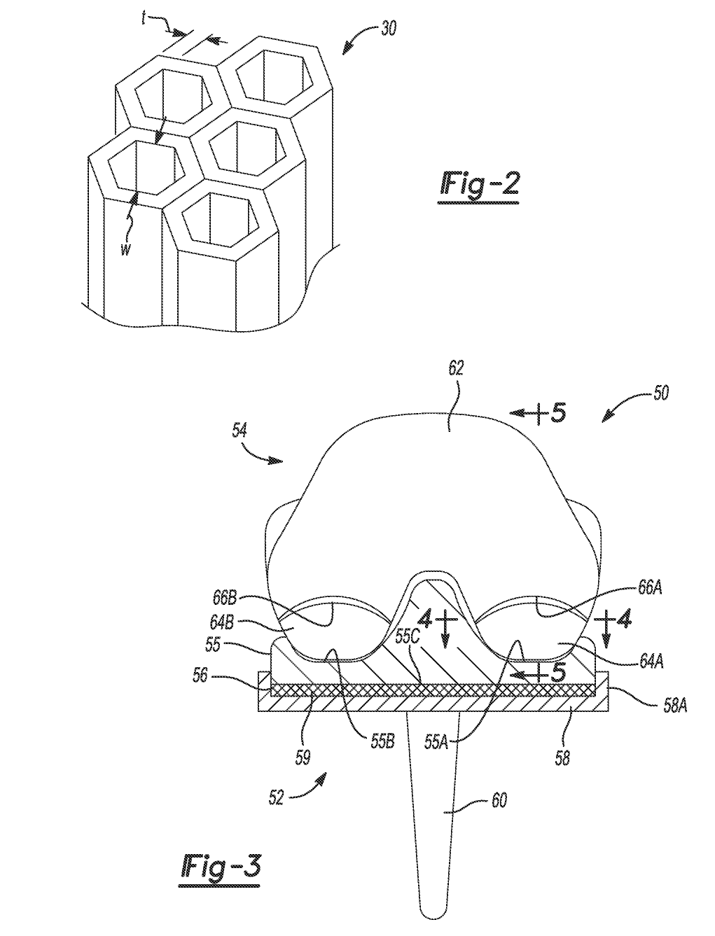

The insert 24 is supported within the acetabular cup 22 by a shock absorbent system or filler 30. The shock absorbent filler 30 is secured to the inner surface 22B of the acetabular cup 22 and to the outer surface 24A of the insert 24. The shock absorbent filler 30 may be secured to the surfaces 22B and 24A using an adhesive or other suitable attachment methods. The shock absorbent filler 30 is made from a deformable, bio-compatible material. The shock absorbent filler 30 is configured to deform under impact or compressive loading between the femur and the pelvis. In a preferred embodiment, the shock absorbent filler 30 has a honey-comb structure, shown in FIG. 2. The deformation characteristics (or ability of the shock absorbent filler 30 to absorb impact loads) of the shock absorbent filler 30 are defined by the material, the thickness “t” of the honeycomb walls, and the width “w” of an individual hexagon. Accordingly, the shock absorbent filler 30 may be tuned to the specific weight of the end user by adjusting “t”, “w”, and the material selection. Further factors, such as expected activity level, gender, and age, may also be accounted for in the tuning of the deformation characteristics of the shock absorbent filler 30. In an alternate embodiment, the shock absorbent filler 30 is made from a biocompatible gel or other deformable or elastic material.

With reference to FIG. 3, a shock absorbent knee prosthesis according to the principles of the present invention is generally indicated by reference number 50. The knee prosthesis 50 includes a tibial component 52 secured to a tibia (not shown), a femoral component 54 secured to the femur (not shown), and a tray insert 55 and a shock absorbent pad 56 disposed between the tibial component 52 and the femoral component 54.

The tibial component 52 includes a tibial plate 58 and a tibial stem 60. The tibial plate 58 is generally planar and flat. The tibial stem 60 extends out perpendicularly from the tibial plate 58. The tibial stem 60 is configured to be secured within the tibia. The tibial stem 60 may be cemented to the tibia or uncemented. The tibial stem 60 is typically metal and may have a surface treatment to facilitate adhesion to the tibia or porous surface treatments to facilitate bone growth. It should be appreciated that the tibial stem 60 may have various other shapes, sizes, and configurations without departing from the scope of the present invention.

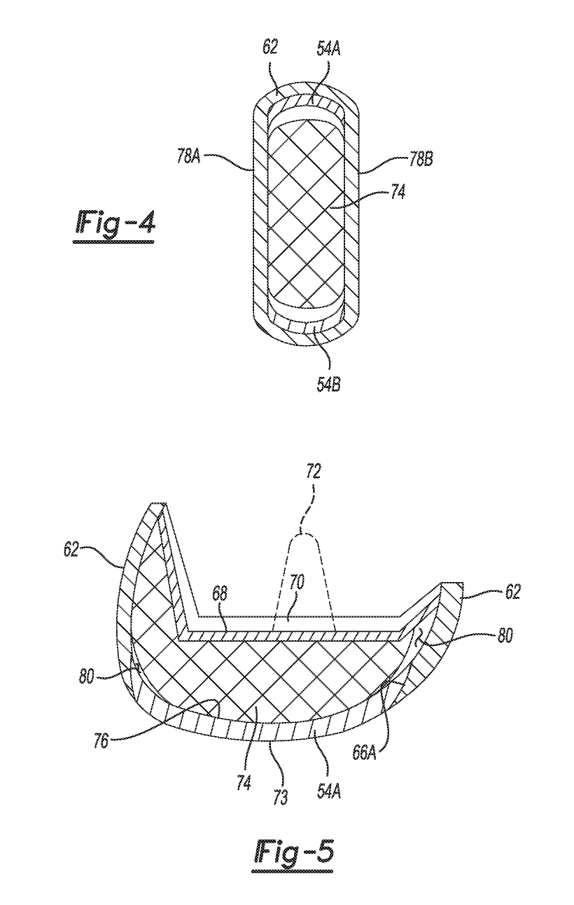

Turning to FIGS. 4 and 5 and with continued reference to FIG. 3, the femoral component 54 includes a support base 62 that supports a pair of moveable condyles 64A and 64B. For example, the support base 62 includes first and second slots 66A and 66B formed therein that are sized to receive the moveable condyles 64A and 64B, respectively. A back plate 68 extends along a back side of the support base 62. The back plate 68 includes a stepped portion 70 configured to interlock with a similarly sized pocket or groove (not shown) formed in the femur. In one example, the stepped portion 70 includes a porous or honeycomb pattern surface to facilitate bone growth. Alternatively, a stem, indicated by dashed lines 72, may extend out from the back plate 68 in place of the stepped portion 70.

The moveable condyles 64A and 64B each have an outer, articulation surface 73 that is contoured to substantially match the contour of the femoral condyles (not shown). The moveable condyles 64A and 64B are precisely machined to slide within the slots 66A and 66B. A shock absorbent material or cushion 74 is sandwiched between the moveable condyles 64A, 64B and the back plate 68. Therefore, the moveable condyles 64A and 64B are able to move within the slots 66A and 66B relative to the support base 62 by deforming the shock absorbent cushion 74.

The shock absorbent cushion 74 may be secured to the moveable condyles 64A, 64B or the back plate 68 using an adhesive or other suitable attachment methods. The shock absorbent cushion 74 extends from an inner surface 76 of each of the condyles 64A and 64B to the back plate. In the example provided, the shock absorbent cushion 74 may also extend between opposing side walls 78A and 78B of the base support 62, where each side wall 78A, 78B cooperates to define the slots 66A and 66B. The shock absorbent cushions 74 and base support 62 may further define gaps or spaces 80 to allow for the movement of the condyles 64A, 64B during impact loading and deformation of the cushion material 74. The shock absorbent cushion 74 is made from a deformable, bio-compatible material. The shock absorbent cushion 74 is configured to deform under impact or compressive loading between the tibia and femur. In a preferred embodiment, the shock absorbent cushion 74 has a honey-comb structure, shown and previously described in FIG. 2. The deformation characteristics (or ability of the shock absorbent cushion 74 to absorb impact loads) of the shock absorbent cushion 74 are defined by the material, the thickness “t” of the honeycomb walls, and the width “w” of an individual hexagon. Accordingly, the shock absorbent cushion 74 may be tuned to the specific weight of the end user by adjusting “t”, “w”, and the material selection. Further factors, such as expected activity level, gender, and age, may also be accounted for in the tuning of the deformation characteristics of the shock absorbent cushion 74. In an alternate embodiment, the shock absorbent cushion 74 is made from a biocompatible gel or other deformable or elastic material.

Returning to FIG. 3, the tray insert 55 is disposed between the femoral component 54 and the tibial component 52. The tray insert 55 includes first and second concave surfaces 55A and 55B and a substantially flat, planar back surface 55C. The first and second concave surfaces 55A and 55B are contoured to match the shapes of the moveable condyles 64A and 64B, respectively. In this way, in a preferred embodiment the moveably condyles 64A and 64B are sized such that no part of the base support 62 contacts the concave surfaces 55A and 55B even as the moveable condyles 64A and 64B move with respect to the base support 62 during impact loads. The tray insert 55 is preferably made from a plastic and provides a surface on which the femoral component 54 may move relative to the tibial component 52.

The shock absorbent pad 56 is sandwiched between the tray insert 55 and the tibial plate 58. In a preferred embodiment, the shock absorbent pad 56 completely covers a surface 59 of the tibial plate 58 and the surface 55C of the tray insert 55. The shock absorbent pad 56 may be secured to the back surface 55C of the tray insert 55 or to the tibial plate 58 using an adhesive or other suitable attachment methods. For example, the tibial plate 58 may include an annular flange 58A that peripherally surrounds the shock absorbent pad 56 and a portion of the tray insert 55. The shock absorbent pad 56 is made from a deformable, bio-compatible material. The shock absorbent pad 56 is configured to deform under impact or compressive loading between the tibia and femur. In a preferred embodiment, the shock absorbent pad 56 has a honey-comb structure, shown and previously described in FIG. 2. The deformation characteristics (or ability of the shock absorbent pad 56 to absorb impact loads) of the shock absorbent pad 56 are defined by the material, the thickness “t” of the honeycomb walls, and the width “w” of an individual hexagon. Accordingly, the shock absorbent pad 56 may be tuned to the specific weight of the end user by adjusting “t”, “w”, and the material selection. Further factors, such as expected activity level, gender, and age, may also be accounted for in the tuning of the deformation characteristics of the shock absorbent pad 56. In an alternate embodiment, the shock absorbent pad 56 is made from a biocompatible gel or other deformable or elastic material.

It should be appreciated that while the above description uses the shock absorbent pad 56 with the shock absorbent cushion 74, either one may be used individually in the shock absorbent prosthesis 50 without departing from the scope of the present invention. In addition, it should be appreciated that the shock absorbent prosthesis 50 may be used in partial knee replacement surgery by bifurcating the prosthesis 50 such that only one moveable condyle 64A or 64B is employed. The partial knee joint prosthesis may include an interlocking system to accommodate a later full knee replacement without removal of the partial knee joint prosthesis.

The description of the invention is merely exemplary in nature and variations that do not depart from the gist of the invention are intended to be within the scope of the invention. Such variations are not to be regarded as a departure from the spirit and scope of the invention.

The following is claimed:

1. A knee joint prosthesis comprising:
a tibial plate;
a femoral component having a moveable condyle in contact with a shock absorbent material, wherein movement of the moveable condyle deforms the shock absorbent material; and
a tray insert disposed between the tibial plate and the moveable condyle.

2. The knee joint prosthesis of claim 1 wherein the femoral component includes a base support that defines a slot, and wherein the moveable condyle is disposed within the slot.

3. The knee joint prosthesis of claim 2 wherein the base support includes a back plate, and the shock absorbent material is in contact with the moveable condyle and the back plate.

4. The knee joint prosthesis of claim 3 wherein the back plate includes a stepped portion having a treated surface configured to engage a matching slot on a femur.

5. The knee joint prosthesis of claim 3 wherein the back plate includes a stem configured to be secured within a femur.

6. The knee joint prosthesis of claim 1 wherein the shock absorbent material is comprised of a material having a honeycomb cross-section.

7. The knee joint prosthesis of claim 6 wherein a deformation characteristic of the shock absorbent material is tuned for a recipient of the knee joint prosthesis by adjusting a thickness of walls of the honeycomb material or a distance between the walls of the honeycomb material.

8. The knee joint prosthesis of claim 1 further comprising a shock absorbent pad disposed between the tibial plate and the tray insert.

9. The knee joint prosthesis of claim 8 wherein the shock absorbent pad completely covers a surface of the tibial plate and a surface of the tray insert.

10. A knee joint prosthesis comprising:
a tibial plate;
a femoral component having a condyle;
a tray insert disposed between the tibial plate and the condyle and having a first surface in contact with the condyle and contoured to match the condyle and an opposing second surface;
a shock absorbent pad disposed between the tibial plate and the tray insert, wherein the shock absorbent pad is in contact with the tibial plate and the second surface of the tray insert, and wherein the shock absorbent pad deforms under a compressive load between the tibial plate and the femoral component.

11. The knee joint prosthesis of claim 10 wherein the shock absorbent pad is comprised of a material having a honeycomb cross-section or a solid material.

12. The knee joint prosthesis of claim 11 wherein a deformation characteristic of the shock absorbent pad is tuned for a recipient of the knee joint prosthesis by adjusting a thickness of walls of the honeycomb material or a distance between the walls of the honeycomb material or by adjusting a density of the solid material.

13. The knee joint prosthesis of claim 12 wherein the deformation characteristic is at least partially defined by a weight of the recipient.

14. The knee joint prosthesis of claim 13 wherein the shock absorbent pad completely covers the tibial plate and the second surface of the tray insert.

15. The knee joint prosthesis of claim 14 wherein the tibial plate includes an annular flange that surrounds an outer diameter of the shock absorbent pad.

16. The knee joint prosthesis of claim 15 wherein the shock absorbent pad is adhered to the tibial plate and the second surface of the tray insert.

17. A knee joint prosthesis comprising:
a tibial plate;
a base support configured to attach to a femur, wherein the base support defines a slot,
a moveable condyle disposed within the slot and able to move relative to the base support, the moveable condyle having an articulation surface and an inner surface;
a back plate disposed opposite the inner surface of the moveable condyle;
a shock absorbent material disposed within the base support and in contact with the inner surface of the moveable condyle and the back plate; and
a tray insert disposed between the tibial plate and the moveable condyle, and
wherein the moveable condyle deforms the shock absorbent material under compressive forces between the tibial plate and the base support.

18. The knee joint prosthesis of claim 17 wherein the shock absorbent material is comprised of a material having a honeycomb cross-section.

19. The knee joint prosthesis of claim 18 wherein a deformation characteristic of the shock absorbent material is tuned for a recipient of the knee joint prosthesis by adjusting a thickness of walls of the honeycomb material or a distance between the walls of the honeycomb material.

20. The knee joint prosthesis of claim 19 wherein the deformation characteristic is at least partially defined by a weight of the recipient.
